(19) United States
(12) Patent Application Publication
(10) Pub. No.: US 2004/0072281 A1
(43) Pub. Date: Apr. 15, 2004

(54) Morphometric tissue or cell preparation

(76) Inventor: Carolin Horstkorte, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/250,782

(22) PCT Filed: Dec. 20, 2001
(86) PCT No.: PCT/EP01/15055

(30) Foreign Application Priority Data
Jan. 9, 2001 (DE) 10101319.1

(51) Int. Cl. G01N 1/30; G01N 33/48; C12N 5/08

(52) U.S. Cl. 435/40.5; 435/366

(57) ABSTRACT

The invention relates to a morphometric tissue or cell preparation, which may be obtained by carrying out the following method steps: a) cells are cultivated in a three-dimensional matrix, over a defined time and under defined conditions, b) after the defined time, the matrix containing the cultivated cells or tissue is incubated with a fixing and staining solution containing an aldehyde and an non-antigen-specific stain.

### DESCRIPTION

The invention relates to a morphometric tissue or cell preparation that can be obtained by the following process stages being implemented: a) cells or tissues are cultivated over a defined period under defined conditions in a matrix, b) after the end of the defined period, the matrix that contains the cultivated cells or tissues is incubated with a staining solution. In addition, the invention relates to a production process for such a preparation as well as uses thereof. Finally, the invention relates to a fixing and staining solution for use in such a process.

A morphometric tissue or cell preparation is treated in a way that allows a detection of the shaping (morphogenesis) of cellular structures or tissue structures. In this connection, it is essential that shapes, for example shapes of cells and/or cell components and/or tissues be made recognizable by means of contrast methods. In this case, detection can be carried out by observation with use of various optical methods, in the simplest case by means of normal light microscopy. Within the framework of detection, a counting of specific shape features, which in most cases are correlated with certain cellular processes, often takes place. The detection can be carried out visually by the human eye, whereby then an observer performs, for example, the identification of desired structures and optionally counting thereof based on the observation of the preparation, optionally through a microscope, or by observing a corresponding imaging. It is simpler and more reliable, however, to automate the detection, for example by recording the (microscopic) image and evaluating by means of a computer-controlled pattern recognition system, which then can also automatically perform a counting. An imaging can be performed photographically or electronically, for example by means of CCD elements. The methodology is used to this end to identify what influence the defined cultivation conditions have on the morphogenesis. The expression of the defined cultivation conditions therefore comprises in particular also non-natural or non-standard conditions, which are characterized by, for example, the addition of one or more prospective active ingredients to a standard medium. It goes without saying that the conditions in any case are to be selected such that virtually immediate apoptosis of all cells in the preparation altogether is not induced. The staining solution is used to emphasize the shaping features of interest in the contrast. The average individual that is skilled in the art can select the dye easily in proportion to the shapes of interest (for example, basic dyes bind to acidic cell structures, such as DNA in the nucleus or RNA in the ribosomes; acidic dyes preferably bind to basic plasma proteins). It is essential in the staining that the dye be bonded securely to (specific) cellular structures such that optional subsequent washing process stages are no longer able to dissolve the dye. The binding sites of the dye then form the stained areas, while areas in which little or no binding sites for the dye are present visualize areas that are stained only slightly or not at all. This is ultimately the contrast mechanism.

A process of the above-mentioned type is known from, for example, the bibliographic reference Leclere, P., Neuroscience: 82, 545-548, 1998. In the process that is known in this respect, ganglion cell explants were treated with a nerve growth factor or cultivated in its presence and then the thus induced neurite growth was analyzed by morphometry. In this case, a staining was performed with use of antibodies that are labeled with dye or that can be labeled with dye. This is consequently an antigen-dependent staining method. Specific stainings of this type are, however, only suitable for quite thin matrices. It is often desirable, however, to use decidedly three-dimensional matrices or cultures whereby then a sufficiently homogeneous antigen-dependent staining is difficult or not at all possible. Moreover, antigen-dependent staining methods are time-consuming and labor-intensive.

From the bibliographic references of Hayakawa, K., et al., Neurosci Lett.: 274, 103-106, 1999, and Carreau, A., Neuropharmacology: 36, 1755-1762, 1997, it is known to perform morphometric analyses by means of the phase contrast microscopy in-situ, i.e., in the unfixed state. The images are, however, very low in contrast, which in particular hampers automatic pattern recognition, if not makes it impossible. In addition, the cultures can degenerate very quickly in this case. In the case of the bibliographic reference first mentioned above, the migration of cells from complex tissues, such as, e.g., in the metastasizing of tumors, is examined. The bibliographic reference that is mentioned second above describes the influence of nerve growth factors on the neurite growth.

However, the invention is based on the technical problem of indicating a morphometric tissue or cell preparation that represents a stable and durable morphogenetic snapshot and that simultaneously can be produced simply in very good contrast.

To solve this technical problem, the invention teaches a morphometric tissue or cell preparation that can be obtained by the following process stages being implemented: a) cells or tissues are cultivated over a defined period under defined conditions in a three-dimensional matrix, b) after the end of the defined period, the matrix that contains the cultivated cells or tissues is incubated with an aldehyde that contains a fixing and staining solution and, optionally, an alcohol, as well as an antigen-unspecific staining agent. A washing process stage, for example with water, can then follow. An antigen-unspecific staining agent is a dye that by itself because of its acid or basic groups, but also by redox processes, can bind unspecifically to molecules or molecule classes of a cell or a tissue. A binding to virtually all structures of cells or a tissue can also take place. Use of antibodies does not take place. The aldehyde and optionally the alcohol produce a reliable fixing of structures of cells or tissue by a large number of immobilizing reactions with mobile molecules, per se, of cells or of tissue, for example in the manner of Mannich reactions. Cell growth is carried out in a three-dimensional matrix, or growth of cell parts is carried out in all three directions in space. In particular, among the latter, preparations are defined whose spatial expansion in all three directions in space is a multiple of the spatial expansion of the cultivated cells, for example the same or more than 2×, preferably more than 10×, to more than 100×. With the invention, it is achieved that the decidedly three-dimensional cell or tissue preparations are stable and permanently fixed and at the same time very contrast-rich images are obtained by means of the simplest methods, for example simple light microscopy. In this case, only a single treatment with the setting and staining solution is necessary, i.e., it is a “one-step” method. Moreover, decidedly three-dimensional preparations can be readily fixed and stained, while a specific staining of such preparations with antibody-based methods is hardly possible, if at all. Decidedly three-dimensional preparations are those that exhibit not only a minimal thickness (section) but rather can exhibit thicknesses of 0.2 μm to 20 mm, especially 0.2 mm to 20 mm.

In a preferred embodiment of the invention, which is of self-evident importance, the setting and staining solution contains water, optionally a water-miscible organic solvent, and common salt. The aqueous common salt solution, especially in the case of a physiological common salt solution, prevents reliable shape changes of cells or tissue, for example caused by osmosis, in the course of the setting and staining.

In particular, the setting and staining solution can contain the following components: A) 0.5-35% by weight, preferably 1-2% by weight, of aldehyde, B) 0.01 to 10% by weight, preferably 0.3-0.6% by weight, of staining agent, C) 0-90% by weight, preferably 30-50% by weight, of one or more water-miscible organic solvents, D) 0-90% by weight, preferably 50-70% by weight, of water, E) 0-5% by weight, preferably 0.1-1% by weight, of common salt, whereby the sum of the portions of components A) to E) always yields 100% by weight, and whereby the ratio of the proportions by weight of components E:D is in the range of 1:1000 to 1:100, and preferably corresponds to the ratio of the proportions by weight of a physiological common salt solution. The invention also independently relates to such a setting and staining solution.

The period of incubation in stage b) can be in the range of 0.5 to 24 hours, preferably in the range of 1 to 10 minutes. The incubation in stage b) can be performed at a temperature of 0° C. to 60° C., preferably from 0° C. to 40° C.

The water-miscible organic solvent can be selected from the group that consists of “C1-10 alkanols with 1-3 OH groups, C1-10 alicyclic alcohols with 1-3 OH groups, C1 to C10 aromatic alcohols and mixtures of such substances.” Ethanol is preferred. The staining agent can be selected from the group that consists of “crystal violet, amido black, cresyl violet, and mixtures of such substances.” The matrix can be selected from the group that consists of “extracted, natural extracellular matrices of different origins, especially of rat tails or pig skin, their main components, especially collagen, laminin, fibronectin, and mixtures of these substances.” The aldehyde can be selected from the group that consists of “C1 to C10 alkane aldehydes, C1 to C10 alicyclic aldehydes, aromatic C1 to C10 aldehydes, and mixtures of such aldehydes.” Formaldehyde and paraformaldehyde are preferred.

The individual cells or complex tissues of the preparation can be made of, for example, tumor cells, nerve cells, glia cells, muscle cells, blood cells or can consist of the above. In the field of finding active ingredients, the cells are optimally (but not necessarily) human cells.

In addition, the invention teaches a process for the production of a morphometric tissue or cell preparation, whereby the following process stages are implemented: a) cells or tissues are cultivated over a defined period under defined conditions in a decidedly three-dimensional matrix, b) after the end of the defined period, the matrix that contains the cultivated cells or tissues is incubated with an aldehyde that contains a fixing and staining solution as well as an antigen-unspecific staining agent. Relative to the process according to the invention, the above comments apply analogously to the preparation.

In addition, the invention teaches the use of a morphometric tissue or cell preparation according to the invention for morphometric analysis of cultivation-condition-induced cellular processes. Expressed in other words, defined conditions are set in stage a) of which a certain morphogenetic effect is expected. The latter is then evaluated and correlated with the defined conditions, optionally semi-quantitatively or quantitatively.

Among the latter also falls the use of a morphometric tissue or cell preparation according to the invention in a process for finding active ingredients, whereby in stage a), the active ingredient is used, whereby then in stage b), an imaging of the cells or the tissue is performed by means of, for example, a light microscope, whereby the imaging that is obtained is preferably evaluated by means of an image recognition system, whereby the result of the evaluation is attributed to the active ingredient, and whereby optionally the process is repeated for various active ingredients, and the results of the evaluation of various active ingredients that are obtained are compared to one another. Finally, quantitative, but at least semi-quantitative information relative to the morphogenetic effect, which is produced by the active ingredients that are examined, is obtained. The cells can be, for example, ganglion cells, especially dorsal, rear root ganglia, whereby the evaluation then comprises, e.g., the detection of newly formed neurites. The cells can also be tumor cells, whereby the evaluation then comprises, e.g., the detection of cells that have migrated out.

Below, the invention is explained in more detail based on a non-limiting example.

### EXAMPLE 1

An explant culture that consisted of dorsal rear root ganglia of the early postnatal rat was set in a decidedly three-dimensional collagen gel (dimensions: diameter 13 mm, height 8 mm). The cultivation was then carried out for several hours in a culture medium that contains a nerve growth factor. The cultivation was completed by suctioning the culture medium from the gel and by the following staining and fixing step.

### EXAMPLE 2

The gel that was obtained in Example 1 was then treated with a filtered fixing and staining solution. The latter contained: 2.42 g of crystal violet, 162 ml of ethanol (100%), 323 ml of distilled water, 16 ml of formaldehyde (35%, remainder water), and 0.8 g of NaCl. The treatment was carried out for 5 minutes at 20° C. Then, it was washed with water.

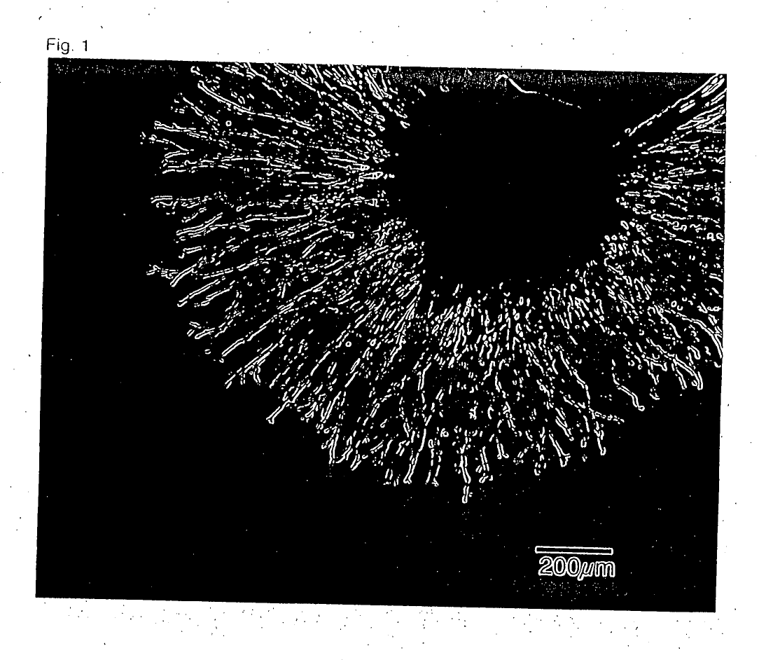

The washed gel was then placed on a light microscope (Zeiss Axiovert, bright field, magnification of the lens 10×), and a detail, as shown in FIG. 1, was produced. In FIG. 1 below, the edge of the explant core is recognizable. In addition, the neurites that are grown from it are detected because of cultivation with the nerve growth factor. It is clear that an extremely contrast-rich figure is obtained that can be analyzed quantitatively in a simple and reliable manner.

1. Morphometric tissue or cell preparation that can be obtained by the following process stages being implemented:
a) Cells or tissues are cultivated over a defined period under defined conditions in a three-dimensional matrix,
b) after the end of the defined period, the matrix that contains the cultivated cells or tissues is incubated with an aldehyde that contains a fixing and staining solution as well as an antigen-unspecific staining agent.

2. Morphometric tissue or cell preparation according to claim 1, whereby the setting and staining solution contains water, optionally a water-miscible organic solvent, and common salt.

3. Morphometric tissue or cell preparation according to claim 1 or 2, whereby the setting and staining solution contains the following components:
A) 0.5-35% by weight, preferably 1-2% by weight, of aldehyde,
B) 0.01 to 10% by weight, preferably 0.3-0.6% by weight, of staining agent,
C) 0-90% by weight, preferably 30-50% by weight, of one or more water-miscible organic solvents,
D) 0-90% by weight, preferably 50-70% by weight, of water,
E) 0-5% by weight, preferably 0.1-1% by weight, of common salt, whereby the sum of the portions of components A) to E) always yields 100% by weight, and
whereby the ratio of the proportions by weight of components E:D is in the range of 1:1000 to 1:100, and preferably corresponds to the ratio of the proportions by weight of a physiological common salt solution.

4. Morphometric tissue or cell preparation according to one of claims 1 to 3, whereby the period of incubation in stage b) is in the range of 0.5 to 24 hours, preferably in the range of 1 to 10 minutes.

5. Morphometric tissue or cell preparation according to one of claims 1 to 4, whereby the incubation in stage b) is performed at a temperature of 0° C. to 60° C., preferably 0° C. to 4° C.

6. Morphometric tissue or cell preparation according to one of claims 1 to 5, wherein the water-miscible organic solvent is selected from the group that consists of “C1-10 alkanols with 1-3 OH groups, C1-10 alicyclic alcohols with 1 to 3 OH groups, C1 to C10 aromatic alcohols and mixtures of such substances.”

7. Morphometric tissue or cell preparation according to one of claims 1 to 6, whereby the staining agent is selected from the group that consists of “crystal violet, amido black, cresyl violet and mixtures of such substances.”

8. Morphometric tissue or cell preparation according to one of claims 1 to 7, whereby the matrix is selected from the group that consists of “natural extracellular matrices of different origins, especially of rat tails or pig skin, the main components of such matrices, especially collagen, laminin, fibronectin, and mixtures of these substances.”

9. Morphometric tissue or cell preparation according to one of claims 1 to 8, whereby the aldehyde is selected from the group that consists of “C1 to C10 alkane aldehydes, C1 to C10 alicyclic aldehydes, aromatic C1 to C10 aldehydes, and mixtures of such aldehydes.”

10. Morphometric tissue or cell preparation according to one of claims 1 to 9, whereby the cells or the tissue are selected from the group that consists of “tumor cells, nerve cells, glia cells, muscle cells, blood cells, tissues that contain such cells, and mixtures of such cells,” preferably of human origin.

11. Process for the production of a morphometric tissue and/or cell preparation, whereby the following process stages are implemented:
a) Cells or tissues are cultivated over a defined period under defined conditions in a decidedly three-dimensional matrix,
b) after the end of the defined period, the matrix that contains the cultivated cells or tissues is incubated with an aldehyde that contains a fixing and staining solution as well as an antigen-unspecific staining agent.

12. Use of a morphometric tissue and/or cell preparation according to one of claims 1 to 10 for morphometric analysis of cultivation-condition-induced cellular processes.

13. Use of a morphometric tissue and/or cell preparation according to one of claims 1 to 10 in a process for finding active ingredients, whereby in stage a), the active ingredient is used, whereby then in stage b), an imaging of the cells or the tissue is performed by means of a light microscope, whereby the imaging that is obtained is preferably evaluated by means of an image recognition system, whereby the result of the evaluation is attributed to the active ingredient and whereby optionally the process is repeated for various active ingredients, and the results of the evaluation of various active ingredients that are obtained are compared to one another.

14. Use according to claim 13, whereby the cells are neurons, especially that consist of dorsal rear root ganglia, and whereby the evaluation comprises the detection of newly formed neurites.

15. Use according to claim 13, whereby the cells are tumor cells and whereby the evaluation comprises the detection of cells that have migrated out.

16. Setting and staining solution for the production of morphometric cell and/or tissue preparations, which contains the following components:
A) 0.5-35% by weight, preferably 1-2% by weight, of aldehyde,
B) 0.01 to 10% by weight, preferably 0.3-0.6% by weight, of staining agent,
C) 0-90% by weight, preferably 30-50% by weight, of one or more water-miscible organic solvents,
D) 0-90% by weight, preferably 50-70% by weight, of water,
E) 0-5% by weight, preferably 0.1-1% by weight, of common salt, whereby the sum of the portions of components A) to E) always yields 100% by weight, and whereby the ratio of the proportions by weight of components E:D is in the range of 1:1000 to 1:100, and preferably corresponds to the ratio of the proportions by weight of a physiological common salt solution.
